(19) United States
(12) Patent Application Publication
(10) Pub. No.: US 2019/0175170 A1
(43) Pub. Date: Jun. 13, 2019

(54) KNOTLESS CLOSURE SUTURE AND TECHNIQUE

(71) Applicant: ARTHREX, INC., Naples, FL (US)

(72) Inventors: Guatam P. Yagnik, Coral Gables, FL (US); Andrew C. Petry, Naples, FL (US); Colin Baker, Naples, FL (US)

(21) Appl. No.: 15/834,615

(22) Filed: Dec. 7, 2017

(51) Int. Cl. A61B 17/04; A61B 17/06; A61L 31/04; A61L 31/06; A61L 31/02; A61L 31/08; A61L 31/10

(52) U.S. Cl. CPC A61B 17/0485; A61B 17/0401; A61B 17/06166; A61B 17/0483; A61L 31/048; A61L 31/06; A61B 2017/0414; A61L 31/088; A61L 31/10; A61B 2017/0445; A61B 2017/044; A61B 2017/0464; A61L 31/022

(57) ABSTRACT

Surgical suturing constructs and methods of tissue fixation are disclosed. The suturing construct is a suture of a small diameter that is provided with a small loop on one end, a splice, and an elongated tapered portion/region at the other end. The splice is located on a region provided with a plurality of laser cuts or protuberances that are locking into the loop when the tapered end is pulled through the loop, to allow the suture to create a cinch stitch around soft tissue.

### BACKGROUND

The present disclosure relates to the field of surgery and, more particularly, to improved sutures and methods of tissue fixation.

### SUMMARY

Suturing constructs and methods for fixation of soft tissue are disclosed. A suturing construct includes a suture with a very small loop on a distal end, and a splice terminating into a tapered portion/region at a proximal end.

The splice portion is provided with multiple laser cuts in one side near the loop at the distal end of the suture. A loader is pre-passed through the loop to load the suture tail back through the loop after passing it through the soft tissue. Once the suture is passed through the soft tissue, the tail is passed through the loop using the loader by folding the tapered end and shuttling it through the loop. The suture is then pulled through until it tightens all the way down. As it tightens, the laser cut roughened portion that is thicker pulls into the loop and prevents the suture from backing up.

Fixation of soft tissue is achieved by using the suture construct passed around or through soft tissue without the need of nitinol wire loops or similar devices. The tapered portion/region of the construct allows the suture construct to be retrieved after being passed through the tissue. After the formation of a cinch stitch and locking with the laser-cut portion near the loop, the suture construct may be secured into bone with additional fixation devices (such as one or more suture anchors). The construct may be employed with knotless or knotted fixation devices.

### BRIEF DESCRIPTION OF THE DRAWINGS

FIG. 1 illustrates an exemplary suturing construct.

FIGS. 2-5 illustrate a method of forming another exemplary suturing construct.

FIGS. 6-8 illustrate subsequent steps of an exemplary method of knotless labral repair with the suturing construct of FIG. 1.

### DETAILED DESCRIPTION

The present disclosure provides methods and suturing constructs for fixation of tissue, for example, soft tissue.

A suturing construct includes a flexible strand with a small loop at its distal end, a tapered proximal end (tapered tail), and a splice region that is obtained from the loop creation and is provided between the small loop and the tapered proximal end. The splice region is provided with multiple laser cuts in one side near the loop at the distal end of the suture. The small loop is formed by splicing but it could be also formed by other methods known in the art, for example, braiding, weaving or gluing.

A shuttling device is pre-loaded onto the loop to load the tapered suture tail back through the loop after passing it through the soft tissue. The flexible strand may be suture, tape, wire, or any flexible material known in the art. The shuttling device may be a suture passing instrument, a suture passer, a shuttle/pull device, a loader, a shuttling wire or any passing instrument, such as FiberLink™ or a Nitinol loop.

Once the flexible strand is passed through the soft tissue, the tapered tail is passed through a loop of the shuttling device by folding the tapered end and shuttling it through the loop. The flexible strand is then pulled through until it tightens all the way down. As it tightens, the laser-cut roughened portion that is thicker pulls into the loop and prevents the flexible strand from backing up.

The flexible strand may be a standard braided coreless suture, may be tapered, and may include three exemplary varying widths throughout its length, with laser cuts in the thickened portion of the suture to create ridges or bumps or barbs. The suture is provided with a small loop to allow the suture to be pulled through until the ridges/bumps/barbs/protuberances engage and lock the suture under tension.

The loop is large enough to allow the tapered end and part of the splice region to pass through it and then lock to the loop, to create a cinch stitch around soft tissue. The flexible strand may be coreless or, in certain applications, may have a core. The loop is formed by splicing but it could be also formed by other methods known in the art, for example, braiding, weaving or gluing.

Fixation of tissue to bone is achieved by using the suturing construct passed around tissue without the need of nitinol wire loops or similar devices. The longer tapered portion/region of the construct allows the suturing construct to be retrieved after being passed through the same portal. After the formation of the cinch stitch and locking of the laser cuts into the loop, the suturing construct may be secured into bone with one or more fixation devices (such as one or two suture anchors). The suturing construct may be used with knotless or knotted fixation devices.

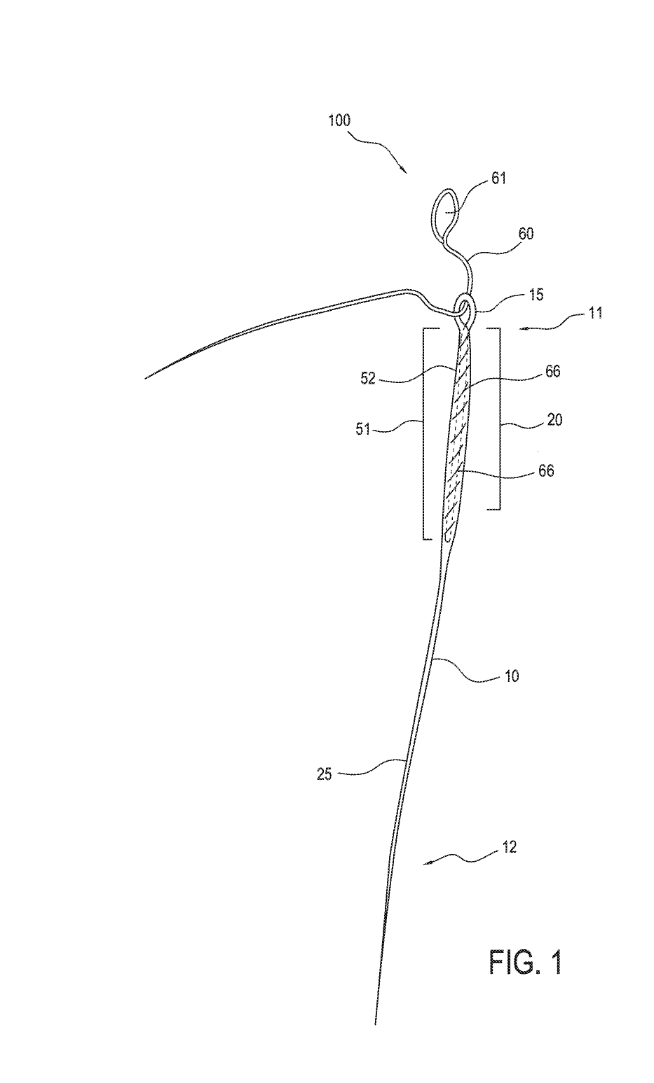

Referring now to the drawings, where like elements are designated by like reference numerals, FIG. 1 illustrates exemplary suturing construct 100 (surgical suture 100; side-to-side knotless suture 100; suture construct 100; knotless closure suture 100) in the form of a flexible strand 10 provided with a small closed loop 15 at one end 11 (for example, distal end) and with a tapered elongated portion/region 25 at other end 12 (for example, proximal end). A splice 52 in splice region 51 is located between the closed loop 15 and the tapered portion 25 and adjacent the closed loop 15. Flexible strand 10 is spliced into itself to form small loop 15 (or a double loop) and splice 52. Small loop 15 is integral to the coreless suture 10. Flexible strand 10 is an exemplary suture that is coreless in the preferred embodiment, but that can also be a suture provided with a core. The tapered portion/region 25 may be stiffened and/or coated/impregnated with a material such as plastic, for example. Tapered portion/region 25 allows for easy locking of the construct.

Suturing construct 100 is also provided with shuttling device 60 which may be a suture passing instrument, loader, shuttling wire or passing instrument, such as FiberLink™ 60 or a Nitinol loop 60 with closed loop or eyelet 61 attached to the flexible strand 50. As shown in FIG. 1, shuttling device 60 is pre-loaded onto closed loop 15 (threaded through the closed loop 15). In particular and exemplary-only embodiments, the flexible strand 10 is a suture strand 10 and the shuttling device 60 is a suture passing device 60. The shuttling device 60 is configured to allow the tapered portion 25 of the flexible strand 50 to pass through eyelet or loop 61 of the shuttling device 60 and through the small loop 15, to form a knotless closed adjustable loop 55 having an adjustable length and perimeter.

Suturing construct 100 also includes a plurality of laser cuts 66 in one side near the loop 15 at the distal end 11 of the flexible strand 10, i.e., on or around portion 60. The laser cuts create bumps and/or barbs and/or ridges that roughen the flexible strand 10 (suture 10) for knotless locking. The laser cuts 66 may have a depth of 0.5-1 mm and may be formed at any angle with the surface of portion 60, for example, at a 45 degree angle with the tangent to surface of portion 60. Laser cuts 66 may be also provided as a plurality of various cuts, each of the plurality being formed at various angles and/or depths relative to top surface of portion 60.

As detailed above, once the flexible strand 10 is passed through the soft tissue, the tail 25 is passed through the loop 61 using the loader 60 by folding the tapered end 25 and shuttling it through the loop 15. The flexible strand 10 is then pulled through until it tightens all the way down. As it tightens, the thicker laser-cut roughened portion 60 with laser cuts 66 pulls into the loop 15 and prevents the flexible strand 10 from backing up. In this manner, flexible closed adjustable loop 55 is locked by the engagement of laser cuts 66 with inner surface of loop 15.

In an exemplary embodiment, the loop 15 has a very small length and/or perimeter to allow the tail 25 to pass through the loop and form a closed, knotless, adjustable loop 55. Loop 15 may have a length of about 10 mm. The tapered portion/region 25 has a length of about 2-3 inches. The overall length of the suture construct may be about 3 to 10 inches, preferably about 5 inches (for exemplary rotator cuff repairs involving attachment of labrum to glenoid). The loop portion 15 is formed by splicing (in the preferred embodiment) but it could be also formed by other methods known in the art, for example, by braiding, weaving or gluing. The loop 15 may be flexible, collapsible and with a fixed perimeter of about 10 mm.

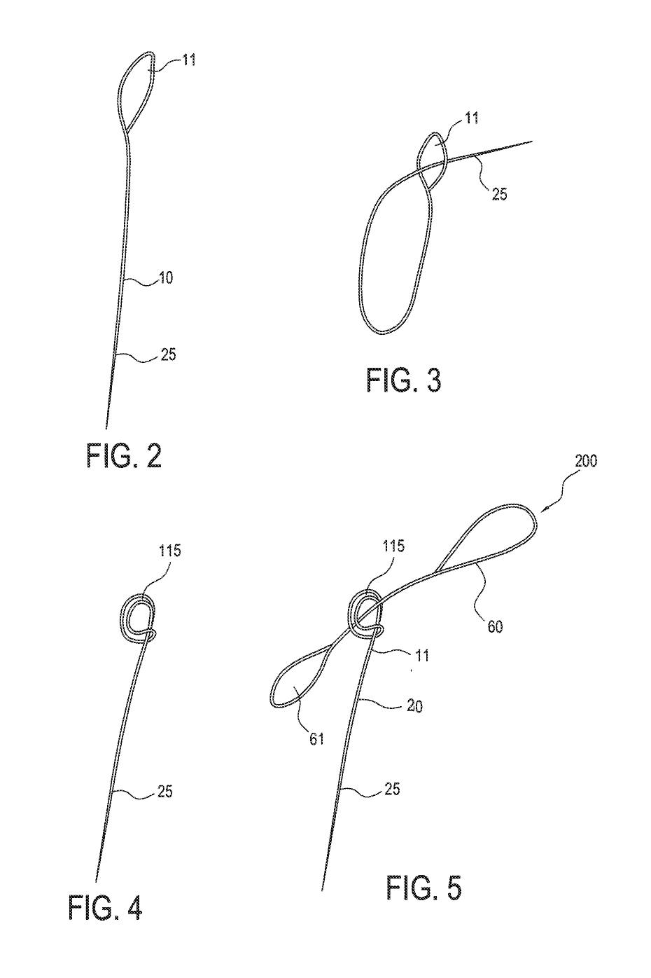

The loop 15 may be also a racking hitch loop. For example, FIGS. 2-5 illustrate a method of forming racking hitch loop 115 of exemplary suturing construct 200 (surgical suture 200; side-to-side knotless suture 200; suture construct 200; knotless closure suture 200) which is similar to the suturing construct 100 of FIG. 1, but differs in that the loop 115 is a racking hitch. Tail 25 (FIG. 2) is passed/loaded through small loop 11 of flexible strand 10, as shown in FIG. 3. The tail is pulled through until a small opening is left (FIG. 4). A suture loader 60 is added, as shown in FIG. 5, to pull the tail through the racking hitch after it is passed through the tissue.

In an exemplary embodiment, the flexible strand 10 of suturing construct 100, 200 is suture formed essentially of a braid such as a FiberWire® CL braid, which is a coreless braid with ultrahigh molecular weight polyethylene (UHMWPE). In another exemplary embodiment, the flexible strand 10 is a suture formed essentially of polyester or similar material. For example, the flexible strand 10 may be formed of polyester yarns (twisted yarns of polyester) or a polyester suture in the form of a coreless braid or sheath which may be a multifilament, braided, knitted, or woven polyester construct, wherein the polyester is provided alone or in combination with any other known suture materials. In an exemplary-only embodiment, the flexible strand 10 is a braided coreless suture with about 100% polyester.

At least one or all of suture sections of suturing construct 100, 200 may be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the final suture construct, knot security, pliability, handleability or abrasion resistance, for example.

The elongated tapered region 25 of the surgical constructs of the present disclosure allows the flexible strand/suture to be pushed through small and very small diameter tubes and cannulations (such as Lasso instruments, for example) yet fix securely with existing fixation devices (such as knotless suture anchors like PushLock® anchors).

Surgical suturing construct 100, 200 may have cross-sections of various forms and geometries, including round, oval, rectangular, or flat, among others, or combination of such forms and geometries. The diameter of construct 100, 200 may be constant or may vary. Tapered region 25 may include a plurality of adjacent tapered regions, each of the regions having a diameter different from those of the adjacent regions. In an exemplary-only embodiment, region 25 may comprise three adjacent regions, each having a taper different from the adjacent regions.

At least a part of the fibers of suturing construct 100, 200 may contain strands of a high strength suture material, such as Arthrex FiberWire® suture disclosed in U.S. Pat. No. 6,716,234, the disclosure of which is incorporated in its entirety by reference herein, with optional colored strands to assist surgeons in distinguishing between various suture lengths.

Surgical suturing construct 100, 200 has applicability to suture applications that may be employed in surgical procedures such as rotator cuff repair, Achilles tendon repair, patellar tendon repair, ACUPCL reconstruction, hip and shoulder reconstruction procedures, and applications for suture used in or with suture anchors. In exemplary embodiments only, the suturing construct 100, 200 may be employed in suture applications that do not involve knot tying, for example, for use with suture anchors (such as PushLock® and/or SwiveLock® suture anchors) or for knotless arthroscopic suture repairs (such as knotless single row rotator cuff repair, or SpeedBridge™ repair using no knots and only suture passing steps), among many others, but with simplified steps as no nitinol wires or similar structures are required.

In an exemplary-only embodiment, construct 100, 200 is employed to bring together a first tissue portion (for example, a first soft tissue) to a second tissue portion (for example, a second soft tissue) in a side-to-side closure repair. The construct is first passed through the first tissue portion. The tail (tapered region 25) is passed through or around the second tissue portion, and then passed through the loop using the shuttling device (loader) by folding the tapered end 25 and shuttling it through the loop. Flexible strand 10 (suture 10) is then pulled until it tightens and achieves the desired tension on closed adjustable loop 55. As the flexible strand 10 tightens, the laser-cut roughened portion 60 of the construct 100, 200 (that is thicker) pulls into the loop 15, 115 and locks cuts 66 to the inner surface of loop 15, 115 (to the flexible strand 10) to prevent the flexible strand 10 from backing up. In an exemplary embodiment, the laser cuts 66 form a plurality of ridges/bumps/barbs/protuberances that engage and lock the suture 10 under tension. The flexible strand may be a standard braided suture that is tapered and that has three varying widths throughout its length.

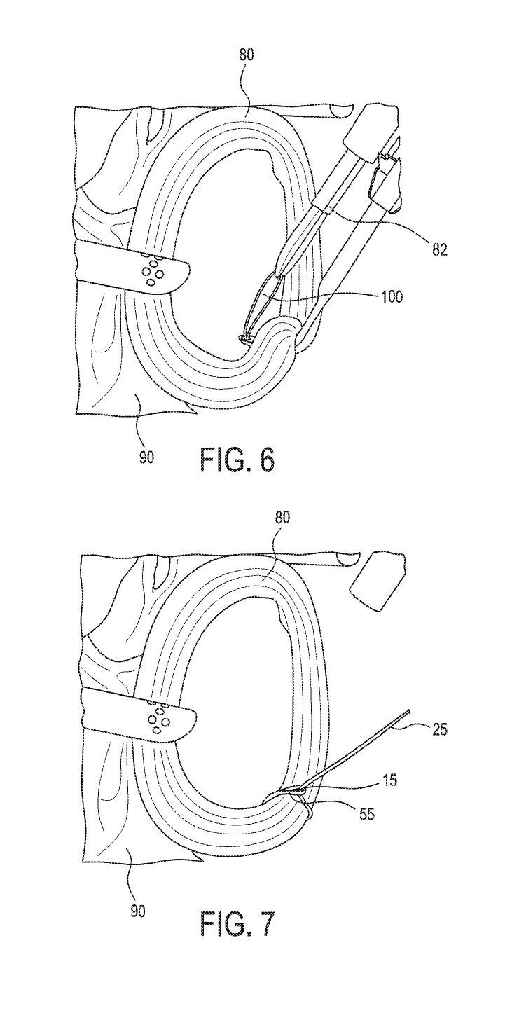

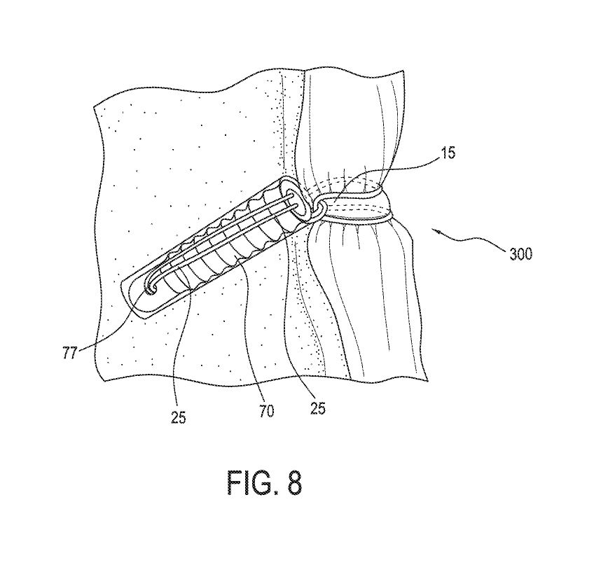

FIGS. 6-8 illustrate an exemplary method of soft tissue repair 300 (FIG. 8) with suturing construct 100. FIG. 6 illustrates a schematic view of a surgical site undergoing a method of fixation of soft tissue to bone (or of soft tissue to soft tissue) by the methods of the present disclosure. In an exemplary embodiment only, the surgical site is the shoulder and the tissue is labrum 80 to be attached to glenoid 90 with exemplary surgical construct 100.

Fixation of soft tissue to bone, such as fixation of labrum to glenoid, typically involves the formation of an incision to access the surgical site and then reattachment of the soft tissue. When soft tissue is attached to bone, the surgeon drills a cavity in the bone and inserts a fixation device such as a bone anchor. Typically, the bone anchor is formed of metal, composite, plastic or bioabsorbable material, and is held in place by threads or by barbs. If an anchor is employed, the anchor typically includes an eyelet through which construct 100 is then threaded/passed.

Fixation of tissue to bone is achieved by using the suturing construct 100 passed around tissue 80 without the need of nitinol wire loops or similar devices. The tapered elongated portion/region 25 allows the suturing construct 100 to be retrieved (with instrument 82 in FIG. 6, for example) after being passed through the same portal. After the formation of cinch stitch 55 (FIG. 7) and the locking of the construct with laser cuts 66 penetrating inner surface area of loop 15, the suturing construct 100 may be secured into bone 90 with one or more fixation devices (such as one or more suture anchors like anchor 70 of FIG. 8) by passing tapered end region 25 through eyelet 77 and then securing the knotless fixation device 70 with suturing construct 100 in bone, to obtain final repair 300 (FIG. 8).

FIG. 8 also illustrates an exemplary knotless fixation device 70 with an eyelet 77 that allows ends of flexible strand 10 (suture 10 of suturing construct 100) to pass therethrough and additionally aid in the fixation of the labrum 80 to the glenoid 90. The flexible strands may be also secured by employing any number of fixation devices, knotless or otherwise, for example two fixation devices such as two knotless fixation devices 70 with two eyelets 77, in lieu of the one fixation device. The technique may be repeated multiple times at different locations in the bone (i.e., multiple cinch stitches at different locations around the labrum).

A method of fixation of a first tissue to a second tissue with suturing construct 100, 200 comprises inter alia the steps of: (i) passing a suturing construct 100, 200 through or around the first tissue, the suturing construct consisting of a length of a flexible strand 10 having a first end and a second end, a small closed loop 15, 115 formed at the first end and having a fixed perimeter, a tapered region 25 formed at the second end, a splice 51 in a splice region 52 located adjacent the first end and the closed loop 15, 115 wherein the splice region (or a region adjacent the loop 115) includes a plurality of laser cuts 66 that prevent the flexible strand 10 from backing up, and a shuttling device 60 with a shuttling loop 61 attached to the flexible strand 10; (ii) passing the tapered end 25 of the flexible strand through shuttling loop 61; and (iii) pulling the shuttling device to allow the flexible strand to form a cinching loop 55 around tissue 80 and to lock laser cuts 66 to an inner surface of loop 15, 115. The method may further comprise the steps of (iv) attaching the suturing construct 100, 200 to a knotless fixation device 70 by threading the tapered end 25 of the suturing construct 100, 200 through an eyelet 77 of the knotless fixation device; and (v) placing the knotless fixation device 70 with the attached suturing construct 100, 200 into a second tissue.

The suturing construct 100, 200 may be employed in surgical procedures such as rotator cuffrepair, Achilles tendon repair, and patellar tendon repair, among many others. Although the invention has been described with reference to a particular application (i.e., fixation of labrum to glenoid in a shoulder repair), it must be understood that the suture construct of the present invention has applicability to any type of repairs (any repair in addition to a shoulder repair) and, thus, the invention is not limited by this exemplary-only embodiment.

In an exemplary embodiment only, the fixation device 70 is a knotless suture anchor such as the two-piece Arthrex PushLock® anchor, disclosed in U.S. Pat. No. 7,329,272, or an Arthrex SwiveLock® anchor, disclosed in U.S. Pat. No. 8,012,174 issued Sep. 6, 2011, U.S. Pat. No. 9,005,246 issued Apr. 14, 2015, and US 2013/0296936 published Nov. 7, 2013, the disclosures of all of which are fully incorporated by reference in their entirety herein.

The suturing construct 100, 200 may be also employed with knotted fixation devices, for example, knotted anchors. Thus, the disclosure is not limited to the use of suturing construct 100, 200 with knotless fixation devices such as fixation device 70, and the disclosure contemplates the use of suturing construct 100, 200 with any type of fixation device, knotless or knotted or combination of knotless and knotted fixation devices. The exemplary constructs of the present disclosure may be employed in various tissue repairs such as knotless rotator cuff repair with the SpeedBridge™ and SpeedFix™ repair techniques, or similar reattachment techniques of soft tissue to bone employing knotless fixation devices for the formation of single, double or multiple row constructs in arthroscopic rotator cuff repairs, or high demand applications like AC joint reconstruction and other areas where tissue pull-through may be a concern.

Flexible strands 10, 60 may be made of any known suture construct, such as multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). FiberWire® suture is formed of an advanced, high-strength fiber material, namely ultrahigh molecular weight polyethylene (UHMWPE), sold under the tradenames Spectra (Honeywell) and Dyneema (DSM), braided with at least one other fiber, natural or synthetic, to form lengths of suture material. The preferred FiberWire® suture includes a core within a hollow braided construct, the core being a twisted yarn of UHMWPE. Flexible strands 10, 60 may be also formed of suture tape, for example, Arthrex FiberTape@, which is a high strength suture tape that is braided and rectangular-like in cross section and as disclosed in U.S. Pat. No. 7,892,256, the disclosure of which is incorporated by reference in its entirety herein. Surgical construct 100, 200 can be used with any type of flexible material or suture known in the art.

The strands may be also formed of a stiff material, or combination of stiff and flexible materials, depending on the intended application. The strands may be also coated and/or provided in different colors. The strands may be also provided with tinted tracing strands, or otherwise contrast visually with the remaining elements of the construct, which could be a plain, solid color, or display a different tracing pattern, for example. Various structural elements of surgical construct 100, 200 may be visually coded, making identification and handling of the suture legs simpler. Easy identification of suture in situ is advantageous in surgical procedures, particularly during arthroscopic surgeries, such as endoscopy and laparoscopy.

Surgical construct 100, 200 may include surgical sutures or similar materials that may be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the suture or tape, knot security, pliability, handleability or abrasion resistance, for example.

Preferably, elongated tapered end 25 may have a very fine end that is coated, impregnated, or stiffened with a material such as plastic, for example.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A suturing construct, comprising:
a coreless suture having a first forming a closed loop integral with the coreless suture, and a second end in the form of a tapered end portion;
a splice region with a splice located between the closed loop and the tapered end portion; and
a shuttling device attached to the closed loop.

2. The suturing construct of claim 1, wherein the coreless suture is a coreless braid or sheath formed of twisted yarns of ultrahigh molecular weight polyethylene that are braided together.

3. The suturing construct of claim 1, wherein the coreless suture is a coreless braid or sheath formed of polyester yarns.

4. The suturing construct of claim 1, wherein the closed loop is formed by splicing the first end through the coreless suture to form the splice and the closed loop.

5. The suturing construct of claim 1, wherein the closed loop is a racking hitch loop.

6. The suturing construct of claim 1, wherein the splice region further comprises a plurality of laser cuts that allow locking of the suturing construct.

7. The suturing construct of claim 1, wherein the tapered end portion has three varying widths.

8. The suturing construct of claim 1, wherein the shuttling device is a Nitinol loop.

9. The suturing construct of claim 1, further comprising a coating provided over the coreless suture.

10. The suturing construct of claim 9, wherein the coating is a silicon or collagen coating.

11. The suturing construct of claim 1, wherein the coreless suture passes through at least one eyelet of a knotless fixation device.

12. The suturing construct of claim 11, wherein the at least one eyelet is located at a most distal end of the knotless fixation device.

13. The suturing construct of claim 11, wherein the knotless fixation device is a swivel anchor or a pushlock anchor.

14. The suturing construct of claim 11, wherein the knotless fixation device comprises an anchor body and an anchor tip rotatably attached to the anchor body, and wherein the anchor body is configured to be inserted over the anchor tip for securing the fixation device into bone.

15. The suturing construct of claim 14, wherein the anchor body is a cannulated interference screw.

16. A method of fixation of soft tissue, comprising:
passing a tapered end of a surgical construct through soft tissue and through a loop at an end opposite to the tapered end of the surgical construct, the surgical construct comprising a flexible strand forming the loop and a splice adjacent the loop and the tapered end, wherein the splice is located on a region with a plurality of laser cuts formed within a surface of the region of the flexible strand; and
pulling on the tapered end to form a knotless adjustable loop around the tissue and to lock at least one of the plurality of laser cuts into the loop.

17. The method of claim 16, further comprising attaching the tapered end to a knotless fixation device and fixing the knotless fixation device and the tapered end.

18. The method of claim 16, wherein the flexible strand is a coreless suture.

19. The method of claim 16, further comprising passing the tapered end through an eyelet of a shuttling device attached to the loop and pulling the shuttling device to allow the tapered end to pass through the loop.

20. The method of claim 16, wherein the soft tissue is rotator cuff.
